(19) United States
(12) Patent Application Publication
(10) Pub. No.: US 2020/0219617 A1
(43) Pub. Date: Jul. 9, 2020

(54) APPARATUS AND METHOD FOR INITIAL INFORMATION GATHERING FROM PATIENTS AT THE POINT OF CARE

(71) Applicant: INTERNATIONAL BUSINESS MACHINES CORPORATION, Armonk, NY (US)

(72) Inventors: Maria Eleftheriou, Mount Kisco, NY (US); Vittorio Castelli, Croton-on-Hudson, NY (US)

(21) Appl. No.: 16/239,324

(22) Filed: Jan. 3, 2019

(51) Int. Cl. G16H 50/20; G16H 10/60; G06F 16/2455

(52) U.S. Cl. CPC G16H 50/20; G06F 16/2455; G16H 10/60

(57) ABSTRACT

Embodiments of the present disclosure relate to dynamically creating a questionnaire for a patient to fill out while waiting to be seen by a healthcare provider. In various embodiments, an initial differential diagnosis may be determined from the dynamic questionnaire. A set of known medical queries and medical information from a set of known medical documents are grouped into categories. A bipartite graph is generated between the categories of medical information and categories of medical queries. A first query is selected and a user is prompted for input of medical data. A first set of candidate diagnoses is determined based on the user input. The remaining queries are classified to determine a ranking. One or more additional queries are selected from the ranked remaining queries, prompting a user for input of additional medical data. A second set of candidate diagnoses is selected based on the additional medical data.

### BACKGROUND

Embodiments of the present disclosure relate to dynamically creating a questionnaire for a patient to fill out while waiting to be seen by a healthcare provider and the dynamic questionnaire may determine an initial differential diagnosis based on information supplied by the patient to better use time with the healthcare provider.

### BRIEF SUMMARY

According to embodiments of the present disclosure, systems, methods of and computer program products for dynamically generating medical queries are provided. In various embodiments, a first clustering algorithm is applied to a set of known medical queries to thereby group the set of known medical queries into categories of medical queries. A second clustering algorithm is applied to a set of known medical documents to thereby group medical information extracted from the set of known medical documents into categories of medical information. A bipartite graph is generated between the categories of medical information and categories of medical queries based on results from the first clustering algorithm and results from the second clustering algorithm. A first query is selected from the set of known medical queries and the first query prompts a user for input of medical data. User input to the first query is received. A first set of candidate diagnoses is determined based on the user input. Remaining queries in the set of medical queries are classified to determine a ranking of the remaining queries based on the first set of candidate diagnoses and whether the remaining queries can be answered by the user. One or more additional queries are selected from the ranked remaining queries and the user is prompted for input of additional medical data. A second set of candidate diagnoses is determined based on the additional medical data, such that the second set of candidate diagnoses is a subset of the first set of candidate diagnoses.

In various embodiments, a system includes a computing node having a computer readable storage medium having program instructions embodied therewith. The program instructions are executable by a processor of the computing node to cause the processor to apply a first clustering algorithm to a set of known medical queries to thereby group the set of known medical queries into categories of medical queries. A second clustering algorithm is applied to a set of known medical documents to thereby group medical information extracted from the set of known medical documents into categories of medical information. A bipartite graph is generated between the categories of medical information and categories of medical queries based on results from the first clustering algorithm and results from the second clustering algorithm. A first query is selected from the set of known medical queries and a user is prompted for input of medical data. User input to the first query is received. A first set of candidate diagnoses is determined based on the user input. Remaining queries are classified in the set of medical queries to determine a ranking of the remaining queries based on the first set of candidate diagnoses and whether the remaining queries can be answered by the user. One or more additional queries are selected from the ranked remaining queries and a user is prompted for input of additional medical data. A second set of candidate diagnoses is determined based on the additional medical data, such that the second set of candidate diagnoses is a subset of the first set of candidate diagnoses.

In various embodiments, a computer program product is provided for dynamically generating medical queries. The computer program product includes a computer readable storage medium having program instructions embodied therewith. The program instructions are executable by a processor to cause the processor to apply a first clustering algorithm to a set of known medical queries to thereby group the set of known medical queries into categories of medical queries. A second clustering algorithm is applied to a set of known medical documents to thereby group medical information extracted from the set of known medical documents into categories of medical information. A bipartite graph is generated between the categories of medical information and categories of medical queries based on results from the first clustering algorithm and results from the second clustering algorithm. A first query is selected from the set of known medical queries and a user is prompted for input of medical data. User input to the first query is received. A first set of candidate diagnoses is determined based on the user input. Remaining queries are classified in the set of medical queries to determine a ranking of the remaining queries based on the first set of candidate diagnoses and whether the remaining queries can be answered by the user. One or more additional queries are selected from the ranked remaining queries and a user is prompted for input of additional medical data. A second set of candidate diagnoses is determined based on the additional medical data, such that the second set of candidate diagnoses is a subset of the first set of candidate diagnoses.

### BRIEF DESCRIPTION OF THE SEVERAL VIEWS OF THE DRAWINGS

FIG. 1 illustrates an exemplary clustering of known medical queries according to various embodiments of the present disclosure.

FIG. 2 illustrates an exemplary clustering of symptoms extracted from medical documents according to various embodiments of the present disclosure.

FIG. 3 illustrates an exemplary bipartite graph between categories of medical information and categories of queries according to various embodiments of the present disclosure.

FIG. 4 illustrates a flow chart illustrating an exemplary method for dynamically generating medical queries according to various embodiments of the present disclosure.

FIG. 5 depicts an exemplary computing node according to various embodiments of the present disclosure.

### DETAILED DESCRIPTION

Often when visiting a healthcare provider (e.g., a physician), a patient is presented with a generic and/or incomplete questionnaire which provides some information to the healthcare provider, at the point of care. In many cases, however, the questionnaire is not provided at all. In various scenarios, this may be caused by support staff (e.g., nurses and/or administrative staff) being overwhelmed and/or overloaded and not being able to adequately screen patients. In other scenarios, some patients may be unable to provide accurate medical data at all, for example, if they are very sick. As a result, medical questionnaires are so generic that the healthcare provider is forced to spend time asking basic medical questions to the patient. For some specialists, this is done by a nurse before talking to the specialist. In most cases, the physician has to spend some of the time gathering basic medical information, which should have been collected in advance.

The average time patients spend with the physicians is about 20 minutes out of an average of 84 minutes that the patient spends at the healthcare provider. Because the physician also asks basic questions, the time of substantive dialogue between patient and physician is significantly limited. At the end of the patient's scheduled time slot with the physician, the physician may curtail a substantive discussion with the patient to move to another patient, thereby leaving the particular patient frustrated as they feel they did not have enough substantive time with the physician.

The remainder of the time (64 mins) is spent waiting at the healthcare provider, interacting with non-physician staff, completing paperwork, billing and similar tasks that are not directly impacting the medical outcome. In fact, research has calculated that the average opportunity cost per visit was $43 and concluded that short visits take a toll on the doctor-patient relationship, which is considered a key ingredient of good care, and may represent a missed opportunity for getting patients more actively involved in their own health. While waiting at the exam room, patients or their caregivers can be provided with the opportunity to answer additional medical questions related to their health conditions. Given the shortage of time with the healthcare provider, the more accurate information that can be collected in advance, the better the medical visit experience and medical outcome may be. In various embodiments, the patient may be using a consumer engagement application on a mobile device or a general purpose computing device (e.g., laptop, tablet, or desktop computer).

In addition, the doctor's time with the patient may be further reduced, given the new fee-for-service payment model, which still dominates U.S. health care and rewards doctors who see patients in bulk.

The systems, methods, and computer program products of the present disclosure relate to dynamically creating a questionnaire for a patient to fill out while waiting to be seen by a healthcare provider. The dynamic questionnaire may provide an initial differential diagnosis based on the questions answered by the patient. Additional questions may be asked of the patient where it is determined that the additional questions can be answered by the patient. The initial differential diagnosis may be revised as additional questions are answered by the patient.

In various embodiments, a first clustering algorithm may be applied to a set of known medical queries. In general, the first clustering algorithm may group similar queries together into bins from which the systems of the present disclosure can draw when forming a dynamic questionnaire. In various embodiments, the first clustering algorithm may be a part of a learning system. In various embodiments, the set of known medical queries may be general questions that relate to all medical fields. For example, patient information (e.g., demographic data) or initial medical intake questions may be collected at the beginning of a medical visit and, thus, these questions may be included in the set of known medical queries. In various embodiments, questions related to prescriptions may be included in the set of known medical queries. For example, a physician may ask if the patient is on any particular medications or if they are allergic to any particular prescriptions. In various embodiments, the known medical queries may be a set of questions that are standard for a particular practice of medicine to ask of patients. For example, patients seeing an orthopedic surgeon may be asked different questions than those patients seeing a nephrologist.

In various embodiments, a second clustering algorithm may be applied to medical information from a set of known medical documents to thereby group the medical queries and the medical information into groups. In various embodiments, the medical documents may be anonymized medical records of visits to a healthcare professional. In general, the second clustering algorithm may group similar medical information together into bins, e.g. databases, from which the systems of the present disclosure can draw when forming a dynamic questionnaire. In various embodiments, the second clustering algorithm may be a part of a learning system that is the same or separate from the first clustering algorithm. In various embodiments, the medical information may be symptoms exhibited by one or more patients. In various embodiments, the medical information may include diagnoses. In various embodiments, the medical information may be demographic information of patients. In various embodiments, the medical information may be prescription information related to a diagnosis. In various embodiments, the medical information may be procedure information related to a medical procedure, such as a surgery. In various embodiments, when clustering symptoms from medical records, the second clustering algorithm may reassign at least one patient symptom from one group to another symptom group.

In various embodiments, natural language processing (NLP) may be applied to the medical documents to thereby extract medical information therefrom. In various embodiments, a description of findings in the medical document may be separated from a diagnostic portion and/or prescriptive portion in the medical document. In various embodiments, separating the description of findings may include annotating the medical document to indicate a plurality of features, extracting the plurality of features, and detecting transitions in the medical document based on the extracted plurality of features. In various embodiments, separating the description of findings includes separating the description of findings into individual sentences, identifying types of medical information (e.g., symptoms, procedures, medications, hedges, negations, etc.), segmenting the medical document into symptoms and medical facts, pairing the symptoms with generated queries, and aligning the generated queries via, e.g., a textual similarity method. One skilled in the art will recognize that any suitable NLP technique may be implemented to process medical documents and extract medical information therefrom for clustering. In various embodiments, the textual similarity method includes word embeddings or cluster of word embeddings.

In various embodiments, the generated queries are automatically generated. In various embodiments, the generated queries are manually generated.

A bipartite graph may be generated between the categories of medical information and categories of medical queries. In various embodiments, the bipartite graph may provide a representation of the connections between the categories of medical information and the categories of questions to determine what categories of medical queries will provide relevant information to the particular categories of medical information. If, for example, the systems of the present disclosure require more information in a particular category of medical information, the system can draw from any of the categories of medical queries that will provide relevant information.

A first query may be selected and a user is prompted for input of medical data. The first query may be selected from an initial predetermined list of medical queries. In various embodiments, the systems of the present disclosure may request information in a particular or, such as, for example, demographic information followed by initial medical information, followed by symptom/disease-related questions. Once the system has received enough information from the user/patient, a first set of candidate diagnoses may be determined. The first set of candidate diagnoses may be a list of possible diagnoses or a single likely diagnosis. However, further information may be required to provide a complete differential diagnosis and eliminate other potential diagnoses.

The remaining queries may be classified to determine a ranking. In various embodiments, the ranking may be based on the queries that the patient is most likely able to answer. In various embodiments, the ranking may be based on the queries that will most likely provide additional useful information to eliminate one or more potential diagnoses. In various embodiments, the ranking may be based on the queries that are most likely to be asked by a particular healthcare provider (e.g., the doctor) or have been previously asked by the healthcare provider. In various embodiments, the ranking may be based on the queries that are generally asked by a healthcare provider in a particular practice/field (e.g., nephrology, orthopedics, ophthalmology, etc.).

One or more additional queries are selected from the ranked remaining queries, prompting a user for input of additional medical data. In various embodiments, the systems of the present disclosure may present additional questions to the user in the order they are ranked from first (highest rank) to last (lowest rank). In various embodiments, the user may be presented questions down the ranking until a valid answer is provided. A second set of candidate diagnoses may selected based on the additional medical data input by the user. Once the additional medical information is entered, the systems of the present disclosure may narrow down the diagnosis to a smaller set of candidate diagnoses or generate an entirely different set of diagnoses if necessary. The second set of candidate diagnoses may be a subset of the first set of candidate diagnoses or may not include any of the diagnoses from the first set.

In various embodiments, the user may be prompted for medical information for which no prior query has been generated. In various embodiments, new queries may be generated from anonymized questionnaires.

In various embodiments, the user may be provided with a set of recommended answers. In various embodiments, the recommended answers are determined from answers to predetermined queries in a database. For example, the recommended answers may be answers that have been provided to the same questions from past patients or may be likely answers to particular, questions. In various embodiments, the user may be provided with a set of queries to ask a healthcare professional. In various embodiments, the user may be presented with answers or questions in a multiple choice format.

In various embodiments the systems of the present disclosure may compare the answers provided by the patient with answers to analogous questions from a known database and if a close match is not found, ask a clarifying question. For example, to the question “how severe is your sinus pain?”, the patient might answer “it really bothers me.” The system would then reply “On a scale of 1 to 10, how would you rate your sinus pain?” or “can you tell me more details?” This ensure that relevant answers are collected for each of the questions.

In various embodiments, a list of questions and/or answers may be crowdsourced from other available non-PHI medical records and compared to the questions asked and/or the answer(s) provided by the patient. In various embodiments, the result of this comparison could be used as an indicator of whether or not sufficient health data was collected for a patient visit. For example, if the patient answer sufficient number of questions in a similar way to the ones listed in EMRs, then the system can stop any further questions. In various embodiments, the systems of the present disclosure may include a predetermined data sufficiency factor and may stop asking the patient for additional medical data once a threshold has been met or surpassed. For example, a predetermined number of answers to questions may be required for each category of information. In this example, questions may be asked of the patient until enough answers are provided to meet the particular threshold.

In various embodiments, published resources for, e.g., ‘how to prepare for a visit for [a disease]” may be mined to extract questions for patients. For example, if symptoms are known, the system may produce a list of diseases. For each disease, the system may provide the patient a list of potential questions to ask the doctor.

In various embodiments, once the user/patient is finished entering medical information into the dynamic questionnaire, the systems of the present disclosure may generate a partial medical record based on the input medical data and additional medical data from the user/patient. Generating a partial medical record for a healthcare provider may save a substantial amount of administrative work for each patient visit, thereby freeing up staff and healthcare providers to focus on their patients.

In various embodiments, the learning system comprises clustering. In general, clustering refers to a task of grouping a set of objects in such a way that objects in the same group (I.e., cluster) are more similar to each other than to those in other groups. In various embodiments, clusters include groups with small distances (e.g., Euclidean) between members, dense areas of the data space, intervals or particular statistical distributions.

In various embodiments, clustering may include connectivity-based clustering (also known as hierarchical clustering). For example, the connectivity-based algorithm may be single-linkage clustering, complete linkage clustering, or unweighted pair group method with arithmetic mean.

In various embodiments, clustering may include centroid-based clustering. In centroid-based clustering, clusters may be represented by a central vector, which may not be a member of the data set. When a specific number of clusters is specified in advance, the centroid-based clustering may include k-means clustering. K-means clustering may include an optimization problem to find k cluster centers such that the squared distances of each data point from the respective assigned cluster center are minimized.

In various embodiments, clustering may include distribution-based clustering. In distribution-based clustering, clusters are defined as objects most likely belonging to the same statistical distribution. For example, a Gaussian mixture model may be used using the expectation-maximization algorithm. In a Gaussian mixture model, the data set may be modeled with a fixed (to avoid overfitting) number of Gaussian distributions that are initialized randomly and whose parameters are iteratively optimized to better fit the data set. This will converge to a local optimum, so multiple runs may produce different results. In order to obtain a hard clustering, objects are often then assigned to the Gaussian distribution they most likely belong to; for soft clusterings, this is not necessary.

In various embodiments, clustering may include density-based clustering. In density-based clustering, clusters are defined as areas of higher density than the remainder of the data set. Objects in these sparse areas—that are required to separate clusters—are usually considered to be noise and border points. Examples of density-based clustering method include DBSCAN and OPTICS. In various embodiments, density-based clustering may include mean-shift, where each object is moved to the densest area in its vicinity, based on kernel density estimation.

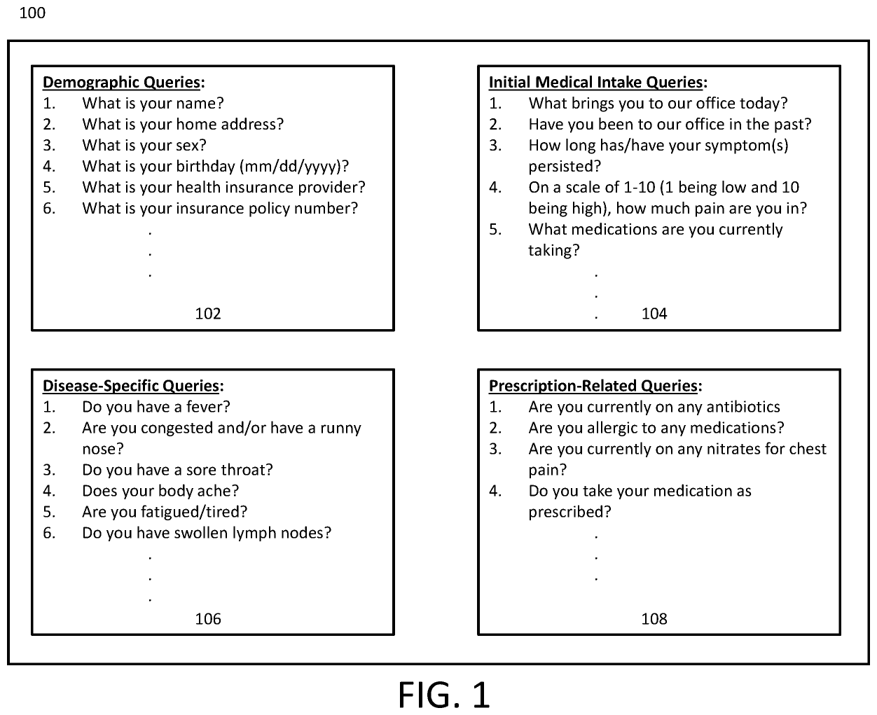

FIG. 1 illustrates an exemplary clustering 100 of known medical queries according to various embodiments of the present disclosure. For example, the clustering of queries may separate queries into demographic queries 102, initial medical intake queries 104, disease-specific queries 106, and prescription related queries 108. One skilled in the art will recognize that any suitable number of clusters/groups may be generated by the clustering systems of the present disclosure. Moreover, any of the categories may include sub-categories. For example, disease-specific queries 106 may include subcategories for, e.g., influenza, common cold, infectious mononucleosis, hepatitis, measles, mumps, rubella, tetanus, tuberculosis, dehydration, various gastrointestinal diseases, cirrhosis, meningitis (viral and/or bacterial), various bone fractures, hernia, eczema, heart disease, pneumonia, diabetes, stroke, chronic obstructive pulmonary disease, various cancers, Alzheimer's, etc.

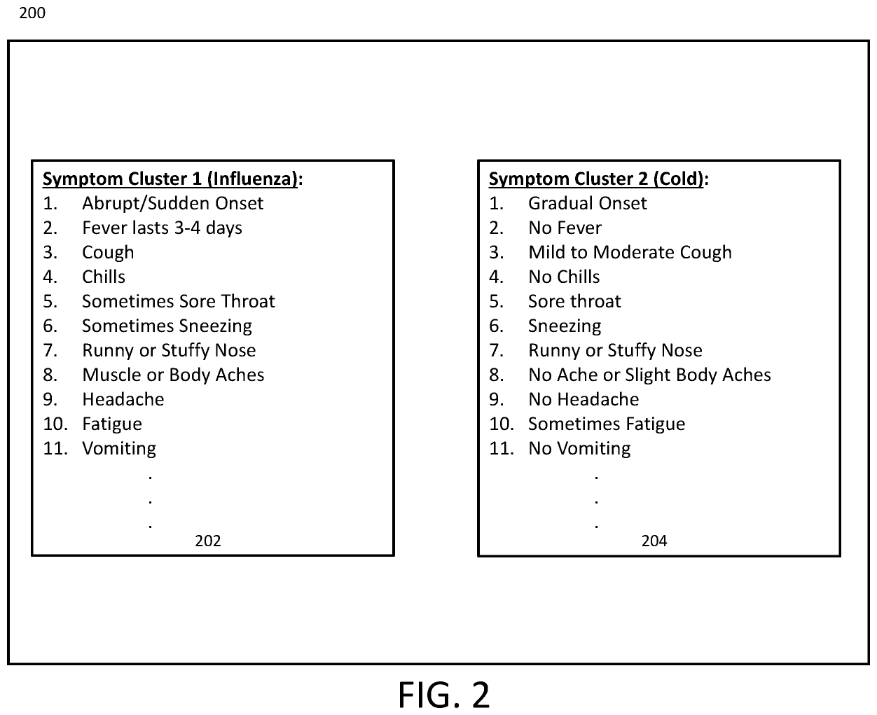

FIG. 2 illustrates an exemplary clustering 200 of symptoms extracted from medical documents according to various embodiments of the present disclosure. In FIG. 2, a first symptom cluster 202 is shown for influenza and a second symptom cluster 204 is shown for a common cold. Symptoms may be clustered based on the final diagnosis information in the medical record and may allow for an initial differential diagnosis when providing a patient with a dynamic questionnaire. For example, if the dynamic questionnaire requests symptoms that the patient is experiencing, and the patient inputs “fever and chills,” the systems of the present disclosure may provide an initial differential diagnosis that “influenza” is a possibility, and that more information/tests may be needed to rule out other conditions. In another example, if the patient inputs “sore throat, no fever, and no chills,” the systems of the present disclosure may provide an initial differential diagnosis that “common cold” is a possibility, and that more information/tests may be needed to rule out other conditions.

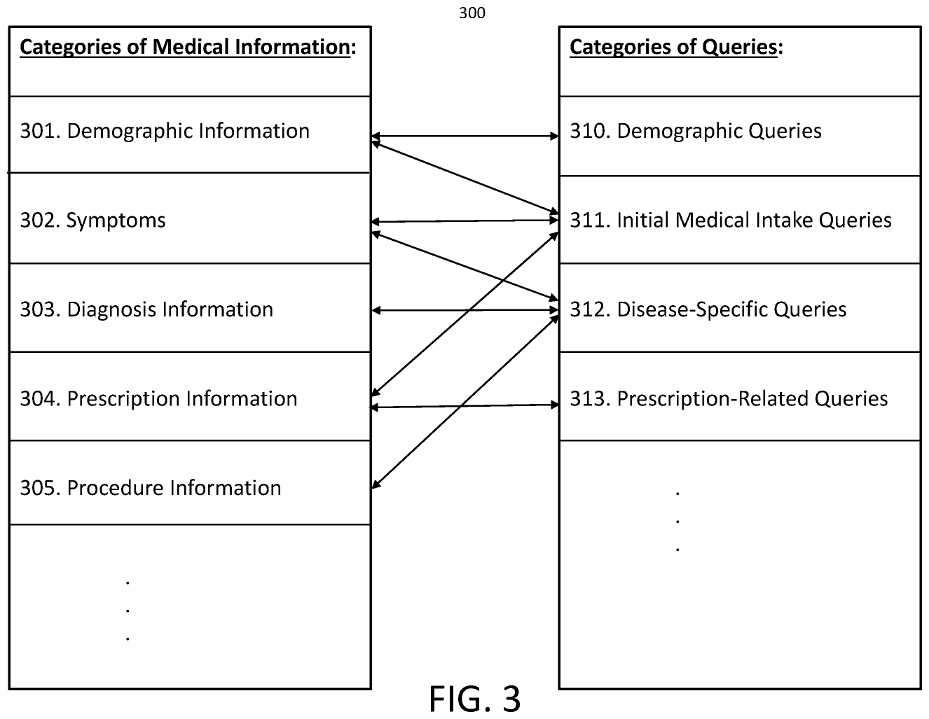

FIG. 3 illustrates an exemplary bipartite graph 300 between categories of medical information and categories of queries according to various embodiments of the present disclosure. The bipartite graph 300 illustrates the connections between several exemplary categories of medical information and the categories of queries that may provide information related to the specific categories of medical information. The exemplary categories of medical information include demographic information 301, symptoms 302, diagnosis information 303, prescription information 304, and procedure information 305. The exemplary categories of queries include demographic queries 310, initial medical intake queries 311, disease-specific queries 312, and prescription related queries 313. As shown in FIG. 3, demographic queries 310 may provide relevant information for the demographic information 301 category of medical information, and thus is connected to one another in the bipartite graph 300. The initial medical intake queries 311 may provide relevant information to demographic information 301, symptoms 302, or prescription information 304, and thus are connected in the bipartite graph 300. The disease-specific queries 312 may provide relevant information to symptoms 302, diagnosis information 303, or procedure information 305, and thus are connected in the bipartite graph 300. Prescription-related queries 313 may provide relevant information to the prescription information 304 category of medical information and, thus, is connected in the bipartite graph 300.

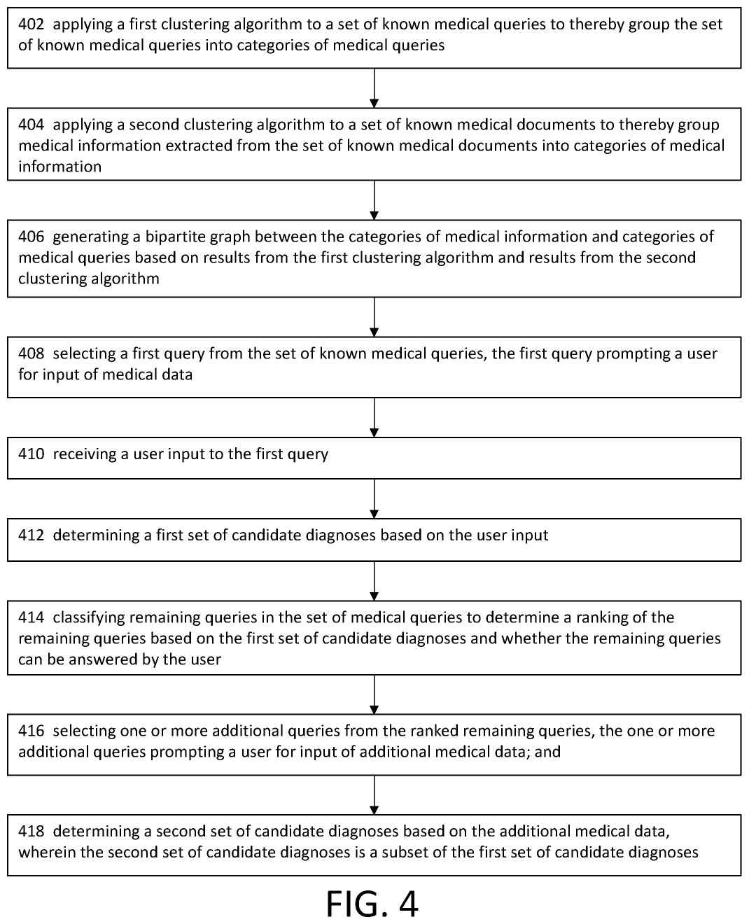

FIG. 4 illustrates a flow chart illustrating an exemplary method 400 for dynamically generating medical queries and performing an initial differential diagnosis. At 402, the method includes applying a first clustering algorithm to a set of known medical queries to thereby group the set of known medical queries into categories of medical queries. At 404, the method further includes applying a second clustering algorithm to a set of known medical documents to thereby group medical information extracted from the set of known medical documents into categories of medical information. At 406, generating a bipartite graph between the categories of medical information and categories of medical queries based on results from the first clustering algorithm and results from the second clustering algorithm. At 408, the method further includes selecting a first query from the set of known medical queries, the first query prompting a user for input of medical data. At 410, the method further includes receiving a user input to the first query. At 412, the method further includes determining a first set of candidate diagnoses based on the user input. At 414, the method further includes classifying remaining queries in the set of medical queries to determine a ranking of the remaining queries based on the first set of candidate diagnoses and whether the remaining queries can be answered by the user. At 416, the method further includes selecting one or more additional queries from the ranked remaining queries, the one or more additional queries prompting a user for input of additional medical data. At 418, the method further includes determining a second set of candidate diagnoses based on the additional medical data, wherein the second set of candidate diagnoses is a subset of the first set of candidate diagnoses.

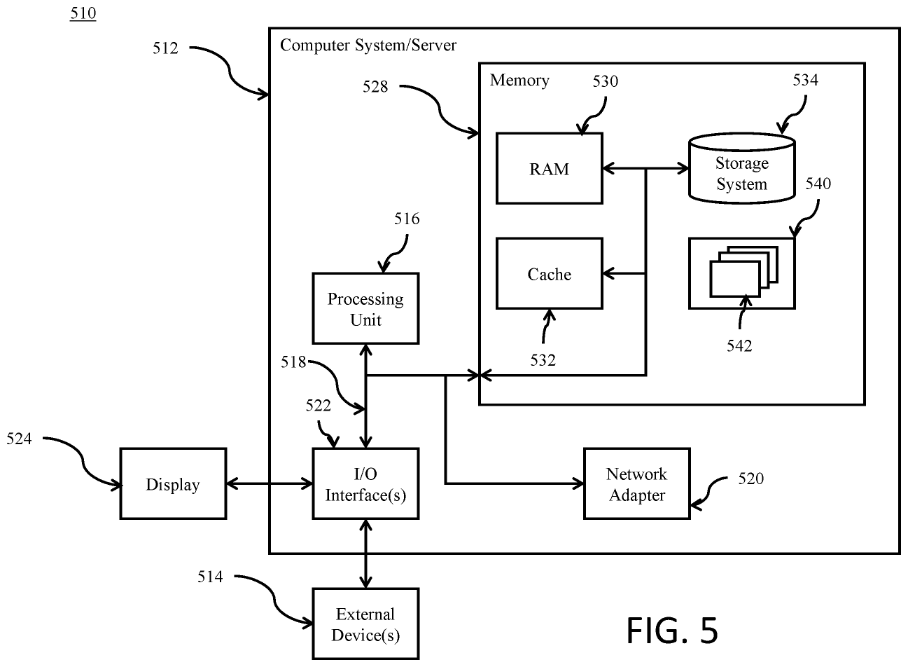

With reference to FIG. 5, a schematic of an example of a computing node is shown. Computing node 510 is only one example of a suitable computing node and is not intended to suggest any limitation as to the scope of use or functionality of embodiments of the invention described herein. Regardless, computing node 510 is capable of being implemented and/or performing any of the functionality set forth hereinabove.

In computing node 510 there is a computer system/server 512, which is operational with numerous other general purpose or special purpose computing system environments or configurations. Examples of well-known computing systems, environments, and/or configurations that may be suitable for use with computer system/server 512 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 512 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 512 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 512 in computing node 510 is shown in the form of a general-purpose computing device. The components of computer system/server 512 may include, but are not limited to, one or more processors or processing units 516, a system memory 528, and a bus 518 that couples various system components including system memory 528 to processor 516.

Bus 518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 512 typically includes a variety of computer system readable media. Such media may be any available media that is accessible by computer system/server 512, and it includes both volatile and non-volatile media, removable and non-removable media.

System memory 528 can include computer system readable media in the form of volatile memory, such as random access memory (RAM) 530 and/or cache memory 532. Computer system/server 512 may further include other removable/non-removable, volatile/non-volatile computer system storage media. By way of example only, storage system 534 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 518 by one or more data media interfaces. As will be further depicted and described below, memory 528 may include at least one program product having a set (e.g., at least one) of program modules that are configured to carry out the functions of embodiments of the invention.

Program/utility 540, having a set (at least one) of program modules 542, may be stored in memory 528 by way of example, and not limitation, as well as an operating system, one or more application programs, other program modules, and program data. Each of the operating system, one or more application programs, other program modules, and program data or some combination thereof, may include an implementation of a networking environment. Program modules 542 generally carry out the functions and/or methodologies of embodiments of the invention as described herein.

Computer system/server 512 may also communicate with one or more external devices 514 such as a keyboard, a pointing device, a display 524, etc.; one or more devices that enable a user to interact with computer system/server 512; and/or any devices (e.g., network card, modem, etc.) that enable computer system/server 512 to communicate with one or more other computing devices. Such communication can occur via Input/Output (I/O) interfaces 522. Still yet, computer system/server 512 can communicate with one or more networks such as a local area network (LAN), a general wide area network (WAN), and/or a public network (e.g., the Internet) via network adapter 520. As depicted, network adapter 520 communicates with the other components of computer system/server 512 via bus 518. It should be understood that although not shown, other hardware and/or software components could be used in conjunction with computer system/server 512. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Artificial neural networks (ANNs) are distributed computing systems, which consist of a number of neurons interconnected through connection points called synapses. Each synapse encodes the strength of the connection between the output of one neuron and the input of another. The output of each neuron is determined by the aggregate input received from other neurons that are connected to it. Thus, the output of a given neuron is based on the outputs of connected neurons from preceding layers and the strength of the connections as determined by the synaptic weights. An ANN is trained to solve a specific problem (e.g., pattern recognition) by adjusting the weights of the synapses such that a particular class of inputs produce a desired output.

Various algorithms may be used for this learning process. Certain algorithms may be suitable for specific tasks such as image recognition, speech recognition, or language processing. Training algorithms lead to a pattern of synaptic weights that, during the learning process, converges toward an optimal solution of the given problem. Backpropagation is one suitable algorithm for supervised learning, in which a known correct output is available during the learning process. The goal of such learning is to obtain a system that generalizes to data that were not available during training.

In general, during backpropagation, the output of the network is compared to the known correct output. An n error value is calculated for each of the neurons in the output layer. The error values are propagated backwards, starting from the output layer, to determine an error value associated with each neuron. The error values correspond to each neuron's contribution to the network output. The error values are then used to update the weights. By incremental correction in this way, the network output is adjusted to conform to the training data.

When applying backpropagation, an ANN rapidly attains a high accuracy on most of the examples in a training-set. The vast majority of training time is spent trying to further increase this test accuracy. During this time, a large number of the training data examples lead to little correction, since the system has already learned to recognize those examples. While in general, ANN performance tends to improve with the size of the data set, this can be explained by the fact that larger data-sets contain more borderline examples between the different classes on which the ANN is being trained.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of dynamically generating medical queries, the method comprising:
applying a first clustering algorithm to a set of known medical queries to thereby group the set of known medical queries into categories of medical queries;
applying a second clustering algorithm to a set of known medical documents to thereby group medical information extracted from the set of known medical documents into categories of medical information;
generating a bipartite graph between the categories of medical information and categories of medical queries based on results from the first clustering algorithm and results from the second clustering algorithm;
selecting a first query from the set of known medical queries, the first query prompting a user for input of medical data;
receiving a user input to the first query;
determining a first set of candidate diagnoses based on the user input;
classifying remaining queries in the set of medical queries to determine a ranking of the remaining queries based on the first set of candidate diagnoses and whether the remaining queries can be answered by the user;
selecting one or more additional queries from the ranked remaining queries, the one or more additional queries prompting a user for input of additional medical data; and
determining a second set of candidate diagnoses based on the additional medical data, wherein the second set of candidate diagnoses is a subset of the first set of candidate diagnoses.

2. The method of claim 1, further comprising prompting a user for medical information for which no prior query has been generated.

3. The method of claim 1, wherein the set of known medical queries comprises questions previously asked by a healthcare professional.

4. The method of claim 1, wherein performing the second clustering algorithm comprises reassigning at least one patient symptom from one group to another group.

5. The method of claim 1, wherein the set of known medical documents are anonymized medical records of visits to a healthcare professional.

6. The method of claim 5, further comprising:
separating a description of findings in the set of known medical documents from a diagnostic portion and/or prescriptive portion.

7. The method of claim 6, wherein separating the description of findings comprises:
annotating the set of known medical documents, wherein the annotations indicate a plurality of features;
extracting the plurality of features; and
detecting transitions in the set of known medical documents based on the extracted plurality of features.

8. The method of claim 7, wherein separating the description of findings further comprises:
separating the description of findings into individual sentences;
identifying a plurality of types of medical information;
segmenting the set of known medical documents into a plurality of symptoms and a plurality of medical facts;
pairing the plurality of symptoms with a plurality of generated queries; and
aligning the plurality of generated queries.

9. The method of claim 8, wherein the plurality of generated queries are automatically generated.

10. The method of claim 8, wherein the plurality of generated queries are manually generated.

11. The method of claim 8, wherein aligning comprises a textual similarity method.

12. The method of claim 11, wherein the textual similarity method comprises word embeddings or cluster of word embeddings.

13. The method of claim 1, further comprising providing the user with a set of recommended answers.

14. The method of claim 13, wherein the recommended answers are determined from answers to predetermined queries in a database.

15. The method of claim 1, further comprising comparing the additional medical data to predetermined medical records to determine a data sufficiency factor.

16. The method of claim 15, further comprising stopping selecting one or more additional queries when the data sufficiency factor is above a threshold.

17. The method of claim 1, further comprising providing the user with a set of questions to ask a healthcare professional.

18. The method of claim 1, further comprising generating a partial medical record based on the medical data and additional medical data.

19. A system comprising:
a computing node comprising a computer readable storage medium having program instructions embodied therewith, the program instructions executable by a processor of the computing node to cause the processor to perform a method comprising:
applying a first clustering algorithm to a set of known medical queries to thereby group the set of known medical queries into categories of medical queries;
applying a second clustering algorithm to a set of known medical documents to thereby group medical information extracted from the set of known medical documents into categories of medical information;
generating a bipartite graph between the categories of medical information and categories of medical queries based on results from the first clustering algorithm and results from the second clustering algorithm;
selecting a first query from the set of known medical queries, the first query prompting a user for input of medical data;
receiving a user input to the first query;
determining a first set of candidate diagnoses based on the user input;
classifying remaining queries in the set of medical queries to determine a ranking of the remaining queries based on the first set of candidate diagnoses and whether the remaining queries can be answered by the user;
selecting one or more additional queries from the ranked remaining queries, the one or more additional queries prompting a user for input of additional medical data; and
determining a second set of candidate diagnoses based on the additional medical data, wherein the second set of candidate diagnoses is a subset of the first set of candidate diagnoses.

20. A computer program product for dynamically generating medical queries, the computer program product comprising a computer readable storage medium having program instructions embodied therewith, the program instructions executable by a processor to cause the processor to perform a method comprising:
applying a first clustering algorithm to a set of known medical queries to thereby group the set of known medical queries into categories of medical queries;
applying a second clustering algorithm to a set of known medical documents to thereby group medical information extracted from the set of known medical documents into categories of medical information;
generating a bipartite graph between the categories of medical information and categories of medical queries based on results from the first clustering algorithm and results from the second clustering algorithm;
selecting a first query from the set of known medical queries, the first query prompting a user for input of medical data;
receiving a user input to the first query;
determining a first set of candidate diagnoses based on the user input;
classifying remaining queries in the set of medical queries to determine a ranking of the remaining queries based on the first set of candidate diagnoses and whether the remaining queries can be answered by the user;
selecting one or more additional queries from the ranked remaining queries, the one or more additional queries prompting a user for input of additional medical data; and
determining a second set of candidate diagnoses based on the additional medical data, wherein the second set of candidate diagnoses is a subset of the first set of candidate diagnoses.
